(19) United States
(12) Patent Application Publication
(10) Pub. No.: US 2026/0144831 A1
(43) Pub. Date: May 28, 2026

(54) PRENATAL PROBIOTIC COCKTAIL TO REDUCE RISK FOR NEURODEVELOPMENTAL DISORDER-ASSOCIATED MALADAPTIVE BEHAVIORAL SYMPTOMS IN CHILDREN

(71) Applicant: Board of Regents, The University of Texas System, Austin, TX (US)

(72) Inventors: Shelly A. Buffington, Galveston, TX (US); Claudia M. Di Gesu, Galveston, TX (US); Lisa M. Matz, Galveston, TX (US); Robert S. Fultz, Galveston, TX (US); Ian J. Bolding, Galveston, TX (US)

(21) Appl. No.: 18/524,521

(22) Filed: Nov. 30, 2023

Related U.S. Application Data
(60) Provisional application No. 63/429,106, filed on Nov. 30, 2022.

(51) Int. Cl. A61K 35/747; A61K 9/00; A61K 35/00; A61K 35/744; A61K 35/745; A61P 1/00

(52) U.S. Cl. CPC A61K 35/747; A61K 9/0053; A61K 35/744; A61K 35/745; A61P 1/00; A61K 2035/115

(57) ABSTRACT

Embodiments of the invention provide a solution for ameliorating microbiome effects on neurodevelopment by developing a probiotic composition for consumption by a female during gestation. In certain aspects the probiotic is a multi-strain probiotic cocktail consisting of immunomodulatory taxa and administered females during pregnancy and lactation.

### RELATED APPLICATIONS

This application claims priority to U.S. Provisional Patent Application Ser. No. 63/429,106 filed Nov. 30, 2022, which is incorporated herein by reference in its entirety.

### STATEMENT REGARDING FEDERALLY FUNDED RESEARCH

None.

### FIELD OF THE INVENTION

Certain embodiments are generally related to the field of medicine and probiotics, and particularly with probiotics and neurodevelopment.

### BACKGROUND

Neurodevelopmental disorders are highly heritable; however, consensus is growing behind a two-hit model in which environmental exposures can promote disease in genetically predisposed individuals. Among environmental factors, those that impact the maternal gut microbiome during pregnancy are emerging as critical modulators of neurodevelopment and long-term behavioral outcomes in offspring. Specifically, disruption, or ‘dysbiosis,’ of the maternal gut microbiome during pregnancy is associated with adverse neurodevelopmental outcomes. Previously, it was shown that maternal high-fat diet (MHFD) in mice induces gut dysbiosis, social dysfunction, and underlying synaptic plasticity deficits in male offspring (F₁).

There is a need for further understanding of the etiology of neurodevelopmental disorders and development of methods and compositions for ameliorating the same.

### SUMMARY

Despite significant advances, the mechanisms underlying the core symptoms of neurodevelopmental disorders, including autism spectrum disorder (ASD) —the prevalence of which is increasing—remain elusive and therapeutic options are limited. Beyond the traditional view of ASD as a purely genetic disorder, the great clinical heterogeneity and phenotypic variability found in ASD patients support a ‘two hit’ model, in which genetic architecture determines the baseline of susceptibility and environmental risk factors can have an additive effect, increasing the chances of reaching the threshold for developing a disease or condition.

Alterations in the maternal environment during fetal development can dysregulate developmental processes, potentially leading to fetal programming of deficits in brain function and behavior. Maternal pre-pregnancy obesity is an important risk factor for multiple adverse long-term health outcomes in offspring. The inventors' investigation of the mechanisms by which maternal obesity contributes to the onset of chronic disorders in the offspring reveals a crucial role for the gut microbiome in mediating the effects of maternal diet on offspring health.

The inventors have investigated whether maternal high-fat diet (MHFD) similarly induces dysbiosis in female offspring (F₁), which was hypothesized to recapitulate the adverse in utero environment experienced by the F₁ males and resulting social dysfunction in the F₂ generation, even in the absence of a maternal (F₁) direct dietary challenge. Metataxonomic sequencing of the fecal microbiome revealed a significant reduction in microbial richness among female F₁ MHFD offspring, with a specific decrease in the abundance of immunomodulatory short-chain fatty acid (SCFA)-producing taxa. Despite recovery of richness in the F₂ generation, F₂ social behavior remained impaired, implicating dysbiosis of the maternal gut microbiome in offspring social deficits. Post-weaning supplementation with probiotic Limosilactobacillus (L.) reuteri was sufficient to rescue F₂ generation social deficits. Unexpectedly, L. reuteri exerted a differential impact on the microbiome of control versus MHFD-descendant F₂ mice, revealing a relative instability of the MHFD lineage microbiome. These differences were particularly strong among females, and this previously unreported heightened responsiveness of the female gut microbiome to probiotic modulation presented an opportunity for intervention: probiotic targeting of the maternal gut microbiome during pregnancy to relieve offspring social dysfunction. Hence, the inventors provide a solution for ameliorating the adverse effects of diet-induced dysbiosis of the maternal microbiome on neurodevelopment by developing a probiotic composition for consumption by a female during gestation. In certain aspects, the probiotic is a 7-strain probiotic cocktail consisting of immunomodulatory taxa administered to females during pregnancy and lactation. Studies described below demonstrate that antenatal targeting of the maternal gut microbiome can restore neurotypical social behavior in offspring. Thus, therapeutically targeting the maternal gut microbiome can improve neurobehavioral outcomes in descendants.

Certain embodiments are directed to multi-strain probiotic compositions. In certain aspects the compositions include a mixture of Bifidobacterium, Lactobacillus, and Streptococcus bacteria and an optional prebiotic component. In certain aspects the mixture includes or consists essentially of Bifidobacterium longum, Lactobacillus acidophilus, Lactobacillus paracasei, Lactobacillus plantarum, Lactobacillus reuteri, and Streptococcus salivarius. The multi-strain probiotic composition can include the probiotic bacteria in a ratio of 1, 2, 3, 4, 5, 6, 7, 8, 9, 10, 11, 12, 13, 14, 15, 16, 17, 18, 19, to 20 Bifidobacterium longum: 1, 2, 3, 4, 5, 6, 7, 8, 9, to 10 Lactobacillus acidophilus: 1, 2, 3, 4, 5, 6, 7, 8, 9, to 10 Lactobacillus paracasei: 1, 2, 3, 4, 5, 6, 7, 8, 9, to 10 Lactobacillus plantarum: 1 to 10 Lactobacillus reuteri: 1, 2, 3, 4, 5, 6, 7, 8, 9, to 10 Streptococcus salivarius. In certain aspects the mixture includes or consists essentially of Bifidobacterium longum ATCC55813, Bifidobacterium longum ssp. Infantis ATCC 15697, Lactobacillus acidophilus ATCC 4356, Lactobacillus paracasei ATCC 25302, Lactobacillus plantarum ATCC 14917, Lactobacillus reuteri ATCC 6475, and Streptococcus salivarius subsp. thermophilus ST28. Certain aspects are directed to a composition wherein the Bifidobacterium longum ATCC55813, Bifidobacterium longum ssp. Infantis ATCC 15697, Lactobacillus acidophilus ATCC 4356, Lactobacillus paracasei ATCC 25302, Lactobacillus plantarum ATCC 14917, Lactobacillus reuteri ATCC 6475, and Streptococcus salivarius subsp. thermophilus ST28 are present in a ratio of 1 to 10:1 to 10:1 to 10:1 to 10:1 to 10:1 to 10:1 to 10, respectively. In certain aspects the Bifidobacterium longum ATCC55813, Bifidobacterium longum ssp. Infantis ATCC 15697, Lactobacillus acidophilus ATCC 4356, Lactobacillus paracasei ATCC 25302, Lactobacillus plantarum ATCC 14917, Lactobacillus reuteri ATCC 6475, and Streptococcus salivarius subsp. thermophilus ST28 are present in a ratio of 1:1:1:1:1:1:1. Each of the bacteria are present in an amount of 1×10³ to about 1×10¹¹ colony forming units (CFU) per gram. In certain aspects the prebiotic is a non-digestible carbohydrate. The composition can be an edible composition. In certain aspects the composition is in the form of a tablet, a powder, a capsule, a solution, or a suspension.

Other embodiments are directed to a nutritional supplement comprising a multi-strain probiotic composition. The multi-strain probiotic compositions including a mixture of Bifidobacterium, Lactobacillus, and Streptococcus bacteria and a prebiotic component. In certain aspects the mixture includes or consists essentially of Bifidobacterium longum ATCC55813, Bifidobacterium longum ssp. Infantis ATCC 15697, Lactobacillus acidophilus ATCC 4356, Lactobacillus paracasei ATCC 25302, Lactobacillus plantarum ATCC 14917, Lactobacillus reuteri ATCC 6475, and Streptococcus salivarius subsp. thermophilus ST28. Each of the bacteria are present in an amount of 1×10³ to about 1×10¹¹ colony forming units (CFU) per gram. In certain aspects the prebiotic is a non-digestible carbohydrate. The composition can be an edible composition. In certain aspects the composition is in the form of a tablet, a powder, a capsule, a solution, or a suspension. The term nutritional supplement refers to a composition comprising a dietary supplement. In some embodiments, nutritional supplements are meal replacements or supplements (e.g., nutrient or energy bars or nutrient beverages or concentrates).

Certain aspects are directed to a nutraceutical comprising a multi-strain probiotic. The multi-strain probiotic nutraceutical can include a mixture of Bifidobacterium, Lactobacillus, and Streptococcus bacteria and an optional prebiotic component. In certain aspects the mixture includes or consists essentially of 2, 3, 4, 5, 6, 7, or more strains of Bifidobacterium longum, Bifidobacterium longum ssp. Infantis, Lactobacillus acidophilus, Lactobacillus paracasei, Lactobacillus plantarum, Lactobacillus reuteri, and Streptococcus salivarius subsp. thermophilus. In certain aspect the mixture includes or consists essentially of 2, 3, 4, 5, 6, or 7 of Bifidobacterium longum ATCC55813, Bifidobacterium longum ssp. Infantis ATCC 15697, Lactobacillus acidophilus ATCC 4356, Lactobacillus paracasei ATCC 25302, Lactobacillus plantarum ATCC 14917, Lactobacillus reuteri ATCC 6475, and Streptococcus salivarius subsp. thermophilus ST28. Each of the bacteria are present in an amount of 1×10³ to about 1×10¹¹ colony forming units (CFU) per gram. In certain aspects the prebiotic is a non-digestible carbohydrate. The composition can be an edible composition. In certain aspects the composition is in the form of a tablet, a powder, a capsule, a solution, or a suspension. The term “nutriceutical” refers to a composition that can serve for nutrition and/or therapeutic purposes.

Other embodiments are directed to methods of treating dysbiosis of a maternal gut biome comprising administering to a female an effective amount of a multi-strain probiotic composition described above.

Still other embodiments are directed to a method of ameliorating the detrimental effects of a high-fat maternal diet on a fetus, infant, or fetus and infant by administering to a mother carrying or given birth to the fetus/infant an effective amount of a multi-strain probiotic composition described above.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1J. Social deficits are prevented in male and female offspring HFD-fed dams treated with a multi-strain probiotic cocktail throughout gestation and lactation. (A) Schematic of breeding scheme to produce MRD−, MRD+MPC−, MHFD−, and MHFD+MPC− descendant F₁ generations with HFD and PC exposure indicated. (B) Timeline of mating, probiotic cocktail administration, tissue collection and behavior. Eppendorf tubes represent time of stool and serum sample collection. (C) Schematic of Crawley's 3C test for sociability and preference for social novelty (3C). (D) MHFD F₁ male offspring show impaired sociability (MRD: t(122)=6.124, p<0.0001; MHFD: t(122)=0.06308, p>0.9999) and (E) preference for social novelty (MRD: t(61)=5.143, p<0.0001; MHFD: t(61)=2.041, p=0.1704) compared to MRD F₁ males; maternal probiotic cocktail prevents MHFD F₁ deficits in sociability (D) (MHFD+MPC: t(122)=3.220, p=0.0066) and preference for social novelty (E) (MHFD+MPC: t(61)=3.111, p=0.0113), and preserves MRD F₁ males sociability (D) (MRD+MPC: t(122)=3.839, p=0.0008) and preference for social novelty (E) (MRD+MPC: t(61)=6.583, p<0.0001). (F-G) Similarly, while MRD F₁ females display normal sociability (F) (MRD: t(68)=3.618, p=0.0023) and preference for social novelty (G) (MRD: t(34)=3.350, p=0.0079), MHFD F₁ females show deficits in sociability (F) (MHFD: t(68)=1.543, p=0.4207) and preference for social novelty (G) (MHFD: t(34)=1.294, p=0.5993). Maternal probiotic cocktail administration effectively prevents impairments in sociability (F) (MHFD+MPC: t(68)=3.849, p=0.0009) and preference for social novelty (G) (MHFD+MPC: t(34)=1.294, p=0.0202) in female descendants of HFD-fed dams, and maintains social preference (F) (MRD+MPC: t(68)=2.783, p=0.0276), (G) (MRD+MPC: t(34)=3.739, p=0.0027), in female descendants of RD-fed dams. (H) Reciprocal social interaction schematic. (I) Comparison of reciprocal social interaction times revealed decreased interaction among MHFD-descendant F₁ male stranger pairs, compared to MRD group, independent of maternal probiotic treatment (MRD vs. MRD+MPC: t(50)=2.307, p=0.0972; MRD vs. MHFD: t(50)=3.210, p=0.0093; MRD vs. MHFD+MPC: t(50)=4.817, p<0.0001; MHFD vs. MHFD+MPC: t(50)=0.9678, p=0.8077). (J) Reciprocal social interaction time is reduced in MHFD F₁ female offspring compared to MRD descendants, and it is partially restored by maternal probiotic supplementation (MRD vs. MRD+MPC: t(26)=0.6111, p=0.9577; MRD vs. MHFD: t(26)=5.835, p<0.0001; MRD vs. MHFD+MPC: t(26)=2.927, p=0.0278; MHFD vs. MHFD+MPC: t(26)=2.706, p=0.0466). Bar graphs show mean±SEM with individual data points.

FIG. 2A-2L. Automated 3C time in chamber data corroborate MHFD-descendant F₂ male social deficits measured by trained observers, related to FIG. 1. 3C ANY-maze automated chamber times for F₁ (A) male and (C) female sociability (Male-MRD: t(122)=5.727, p<0.0001; MHFD: t(122)=0.8852, p=0.8501; MRD+MPC: t(122)=5.074, p<0.0001; MHFD+MPC: t(122)=2.646, p=0.0364) (Female-MRD: t(68)=3.313, p=0.0059; MHFD: t(68)=1.293, p=0.5911; MRD+MPC: t(68)=2.984, p=0.0157; MHFD+MPC: t(68)=2.471, p=0.0624) and social novelty (B) (Male-MRD: t(122)=2.997, p=0.0131; MHFD: t(122)=2.528, p=0.0501; MRD+MPC: t(122)=2.168, p=0.1223; MHFD+MPC: t(122)=0.4126, p=0.9896) (D) (Female-MRD: t(68)=2.720, p=0.0327; MHFD: t(68)=2.517, p=0.0556; MRD+MPC: t(68)=2.618, p=0.0428; MHFD+MPC: t(68)=1.593, p=0.3890) respectively. Social Preference Index for (E) male and (G) female sociability (Males-MRD vs. MHFD: t(61)=2.493, p=0.0601; MRD vs. MRD+MPC: t(61)=0.5395, p=0.9722; MRD vs. MHFD+MPC: t(61)=2.581, p=0.0482; MHFD vs. MHFD+MPC: t(61)=0.007439, p>0.9999), (Female-MRD vs. MRD MPC: t(34)=0.4343, p=0.9877; MRD vs. MHFD: t(34)=0.9249, p=0.8338; MRD vs. MHFD MPC: t(34)=0.03511, p>0.9999; MHFD vs. MHFD MPC: t(34)=1.058, p=0.7565) and social novelty (F, H) (Males-MRD vs. MHFD: t(61)=1.863, p=0.2433; MRD vs. MRD+MPC: t(61)=0.5883, p=0.9620; MRD vs. MHFD+MPC: t(61)=1.321, p=0.5725; MHFD vs. MHFD+MPC: t(61)=0.9412, p=0.6667), (Female-MRD vs. MRD MPC: t(34)=0.2823, p=0.9976; MRD vs. MHFD: t(34)=0.1289, p=0.9999; MRD vs. MHFD MPC: t(34)=0.7080, p=0.9290; MHFD vs. MHFD MPC: t(34)=0.5136, p=0.9771). While reciprocal social (I) contact duration (Males-MRD vs. MRD+MPC: t(50)=2.122, p=0.1464; MRD vs. MHFD: t(50)=0.7286, p=0.9209; MRD vs. MHFD+MPC: t(50)=3.811, p=0.0015; MHFD vs. MHFD+MPC: t(50)=2.750, p=0.0327), is decreased by MHFD and probiotic cocktail in F₁ males, (J) maternal probiotic treatment determines an increase in number of contacts in MHFD-descendants (Male-MRD vs. MRD+MPC: t(50)=0.1503, p=0.9998; MRD vs. MHFD: t(50)=3.367, p=0.0059; MRD vs. MHFD+MPC: t(50)=0.9047, p=0.8425; MHFD vs. MHFD+MPC: t(50)=2.846, p=0.0254). (K) MHFD reduces contact duration in F₁ females, only partially prevented by maternal probiotic administration (Female-MRD vs. MRD MPC: t(26)=1.837, p=0.2763; MRD vs. MHFD: t(26)=4.446, p=0.0006; MRD vs. MHFD MPC: t(26)=2.899, p=0.0297; MHFD vs. MHFD MPC: t(26)=1.347, p=0.5688). (L) No statistically significant differences in number of contacts (Female-MRD vs. MRD MPC: t(26)=1.164, p=0.6916; MRD vs. MHFD: t(26)=0.1282, p=0.999; MRD vs. MHFD MPC: t(26)=2.899, p=0.8679; MHFD vs. MHFD MPC: t(26)=1.056, p=0.768) are observed between cohorts. Bar graphs show mean±SEM with individual data points.

FIG. 3A-3L. Neither maternal diet or probiotic treatment alter anxiety-like behavior and locomotor activity in male and female F₁ offspring. (A) Open field (OF) schematic. No statistically significant differences in OF (B) distance traveled (MRD vs. MHFD: t(14)=0.5858, p=0.9649; MRD vs. MRD+MPC: t(14)=0.5760, p=0.9670; MHFD vs. MHFD+MPC: t(14)=1.386, p=0.5639; MRD vs. MHFD+MPC: t(14)=0.6376, p=0.6376) (C) speed (MRD vs. Male MHFD: t(14)=0.5726, p=0.9677; MRD vs. MRD+MPC: t(14)=0.5930, p=0.9634; MHFD vs. MHFD+MPC: t(14)=1.346, p=0.5899; MRD vs. MHFD+MPC: t(14)=0.6146, p=0.9585) or (D) time spent in center (MRD vs. MHFD: t(14)=0.8029, p=0.8984; MRD vs. MRD+MPC: t(14)=0.1050, p>0.9999; MHFD vs. MHFD+MPC: t(14)=1.198, p=0.6852; MRD vs. MHFD+MPC: t(14)=1.936, p=0.2627) are observed between F₁ male cohorts, regardless of maternal diet or probiotic treatment. Similarly, no statistically significant differences in OF (E) distance traveled (MRD vs. MHFD: t(9)=1.037, p=0.7949; MRD vs. MRD+MPC: t(9)=0.5820, p=0.9673; MHFD vs. MHFD+MPC: t(9)=0.9864, p=0.8212; MRD vs. MHFD+MPC: t(9)=0.2780, p=0.9980), (F) speed (MRD vs. MHFD: t(9)=1.023, p=0.8019; MRD vs. MRD+MPC: t(9)=0.5870, p=0.9663; MHFD vs. MHFD+MPC: t(9)=0.9684, p=0.8303; MRD vs. MHFD+MPC: t(9)=0.2579, p=0.9985) or (G) time spent in center (MRD vs. MHFD: t(9)=0.4699, p=0.9849; MRD vs. MRD+MPC: t(9)=0.6128, p=0.9608; MHFD vs. MHFD+MPC: t(9)=2.009, p=0.2695; MRD vs. MHFD+MPC: t(9)=1.123, p=0.7467) are found between females. (H) Schematic of the 3C habituation stage. Consistently, no differences in distance travelled (I) (MRD vs. MHFD: t(15)=0.5937, p=0.9630; MRD vs. MRD+MPC: t(15)=0.1036, p>0.9999; MHFD vs. MHFD+MPC: t(15)=0.8267, p=0.8879; MRD vs. MHFD+MPC: t(15)=0.2396, p=0.9988) or speed (J) (MRD vs. MHFD: t(15)=0.5940, p=0.9630; MRD vs. MRD+MPC: t(15)=0.08428, p>0.9999; MHFD vs. MHFD+MPC: t(15)=0.8557, p=0.8752; MRD vs. MHFD+MPC: t(15)=0.2723, p=0.9980) are observed between F1 male cohorts in the habituation phase of the 3C task. Likewise, no differences in distance travelled (K) (MRD vs. MHFD: t(33)=0.3774, p=0.9928; MRD vs. MRD+MPC: t(33)=0.1065, p=0.7526; MHFD vs. MHFD+MPC: t(33)=1.376, p=0.5438; MRD vs. MHFD+MPC: t(33)=0.2133, p=0.9992) or (L) speed (MRD vs. MHFD: t(33)=0.3921, p=0.9916; MRD vs. MRD+MPC: t(33)=0.1072, p=0.7479; MHFD vs. MHFD+MPC: t(33)=1.362, p=0.5533; MRD vs. MHFD+MPC: t(33)=0.1931, p=0.9995) are found between females in the 3C habituation phase. Bar graphs show mean±SEM with individual data points.

FIG. 4. Probiotic-mediated manipulation of the maternal microbiota during pregnancy prevents social dysfunction in the offspring of HFD-fed dams. Administration of a multi-strain probiotic cocktail during pregnancy and lactation prevents social dysfunction in the offspring of HFD-fed dams, likely by boosting SCFA availability. The immunomodulatory activity of SCFAs counteract diet-induced pathological inflammation and promote normal epigenetic programming in the fetal brain.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

In recent years, epigenetic studies suggest a role for microbiome-derived metabolites in driving abnormal epigenomic programming in the offspring brain; however, the precise mechanisms by which diet-induced dysbiosis of the maternal gut microbiome impairs offspring neurodevelopment remain poorly understood. The discovery of the underlying mechanism by which pre-pregnancy obesity predisposes affected children to neurodevelopmental disorders would promote the development of predictive biomarkers and prophylactic treatments, including those targeting the maternal microbiome, thereby meeting a yet unrealized opportunity for providing early-life preventative care to a readily identifiable vulnerable population during defined critical periods.

Multiple studies have shown that the gestational period is associated with a significant shift in the types and abundance of commensal bacteria in the maternal gut, with significant differences between trimesters (Koren et al., Cell 150, 470-80, 2012; Turjeman et al., Current Opinion in Endocrine and Metabolic Research 18, 133-38, 2021; Yang et al., NPJ Biofilms Microbiomes 6, 32, 2020). Remodeling of the maternal gut microbiome during pregnancy is driven by the physiological changes in hormonal levels, immunity, and metabolic function occurring in the female body, and are thought to sustain optimal fetal development. At the same time, microbial communities of the maternal gut may influence pregnancy-related immune and metabolic adaptations in the mother, as well as gut barrier function, through the actions of a variety of bioactive microbially-derived metabolites. However, the precise mechanisms by which pregnancy shapes the maternal microbiome, and how the microbiome shapes maternal physiology, and, thereby, fetal and early post-natal development have not been fully elucidated, and further studies incorporating multi-OMIC approaches are warranted (Ghaemi et al., Bioinformatics 35, 95-103, 2019).

The remodeling of the gut microbiome during pregnancy is particularly evident in the third trimester, which is characterized by an increase in total bacterial load and a reduction in microbial richness. Additionally, there is an increase in the relative proportion of opportunistic pathogens, which, in the context of the mother-to-infant vertical transmission of the gut microbiota (Ferretti et al., Cell Host Microbe 24, 133-45 e135, 2018), might be involved in the development of the offspring immune system. In a recent study, increased levels of progesterone in the third trimester have been shown to directly modulate Bifidobacteria abundance in the maternal gut (Nuriel-Ohayon et al., Cell Rep 27, 730-36 733, 2019). Bifidobacterium plays an important role the developing infant microbiota (Koenig et al., Proc Natl Acad Sci USA 108 Suppl 1, 4578-85, 2011), and its increased abundance in the maternal gut near the end of the pregnancy might facilitate its transmission to the offspring. Late pregnancy-specific microbiota has been associated with maternal weight gain, inflammation, and insulin resistance in mice. Intriguingly, while these conditions are generally considered pathological, typically found in individuals affected by metabolic syndrome or diabetes, in the case of gestation they might be required to ensure proper fetal growth and development (Mor and Cardenas, Am J Reprod Immunol 63, 425-33, 2010). Given the central role of maternal gut microbiota in offspring development, it is crucial to investigate how altered maternal host-microbiota interactions may impact the maternal environment and fetal-placental unit, thus triggering fetal programming events which ultimately increase susceptibility to disease.

Diet is considered the primary determinant of gut microbiota composition and function (David et al., Nature 505, 559-63, 2014; Rothschild et al., Nature 555, 210-15, 2018), and imbalanced maternal dietary patterns, such as high-fat diet (HFD), alter both the maternal and offspring microbiome, with negative consequences for offspring development and long-term health outcomes, also in the context of brain function.

For these reasons, recent studies have begun to unravel the effect of maternal high-fat diet (MHFD) on the remodeling of the maternal gut microbiome during gestation. Studies in mice showed that HFD administration during the periconceptional and gestational periods induced significant alterations in microbiome composition compared to dams fed a regular diet, with a remarkable increase in microbial species involved in the biosynthesis of ketone bodies, fatty acid, vitamins, and bile acids (Gohir et al., J Physiol 597, 3029-51, 2019). In subsequent studies, it was shown that HFD determined a reduction in the abundance of short-chain fatty acids (SCFAs) and SCFA-producing bacteria, including Lachnospiraceae and Ruminococcaceae, which was associated with increased levels of pro-inflammatory markers lipopolysaccharides (LPS) and tumor necrosis factor (TNF), loss of intestinal epithelial barrier integrity, and placental hypoxia (Gohir et al., J Physiol 597, 3029-51, 2019). These alterations were accompanied by increased placental inflammation and impairment in both the maternal and offspring glucose metabolism (Wallace et al., Sci Rep 9, 17621, 2019). Changes observed in preclinical animals are highly translational, given that human studies have shown that overweight pregnant women harbor a distinct microbiota composition compared to those of normal weight (Collado et al., Am J Clin Nutr 88, 894-99, 2008) associated with alterations in several metabolic hormones and pregnancy metabolism (Gomez-Arango et al. Diabetes 65, 2214-23, 2016). Taken together, these studies highlight the crucial role of the maternal microbiome in the pregnancy-associated metabolic adaptations and make the case for more extensive investigation into how environmental-induced alterations in maternal gut microbial ecology can influence these important physiological changes and their consequences for fetal development and programming of disease, including neurodevelopmental disorders.

Intriguingly, therapeutic modulation of the maternal gut microbiome during pregnancy through pre- or probiotic supplementation may represent an effective strategy to shift the maternal gut microbial ecology and provide a healthier in utero environment, thus ensuring proper fetal development and lowering the risk negative health outcomes. Probiotics have the potential to remedy dysbiosis of the gut microbiome through a variety of mechanisms, including restoration of intestinal barrier integrity, modulation of the immune response, and secretion of antimicrobial compounds (van Zyl et al., Gut Microbes 12, 1831339, 2020), and are generally regarded as safe (GRAS) to administer during pregnancy (Allen et al., J Nutr 140, 483-88, 2010; Sheyholislami and Connor, Nutrients 13, 2021). In a recent study, maternal probiotic supplementation during gestation and lactation was found to have a protective effect against direct proinflammatory insults in the offspring, by promoting intestinal barrier integrity and modulating inflammatory response (Yu et al., PLoS One 15, c0237182, 2020).

A limited number of studies have investigated daily probiotic administration in obese pregnant women or animal models (reviewed in Wiedmer and Herter-Acberli, Front Nutr 9, 819882, 2022), with some of them showing increased gut microbial diversity (Halkjaer et al., Curr Dev Nutr 4, nzaa095, 2020), with limited effects on metabolic parameters (Callaway et al., Diabetes Care 42, 364-71, 2019). Promisingly, a randomized double-blinded Danish study investigating the efficacy of a multi-strain probiotic versus placebo-control to positively impact blood glucose, gestational weight gain, and reduce risk of gestational diabetes mellitus in obese pregnant women demonstrated >80% adherence to the probiotic regimen and an increase in alpha diversity of the probiotic group over time. In contrast, no increase in alpha diversity was observed in the placebo control group.

Both single-strain and multi-strain probiotic treatment have been used to target gut-brain-behavior interactions and ameliorate or prevent neuropsychiatric outcomes in human and animal studies (reviewed in Joseph and Law, Neurosci Biobehav Rev 99, 160-97, 2019; and Le Morvan de Sequeira et al., Nutrients 14, 2022). However, there is a paucity of studies investigating the efficacy of maternal probiotic supplementation in counteracting the detrimental effects of HFD-induced dysbiosis of gut microbiome on offspring neurodevelopment. A recent study in CD-1 IGS mice (Radford-Smith et al., Proc Natl Acad Sci USA 119, 2022) showed that multi-strain probiotic (Bio-Kult Advanced® containing Bifidobacterium spp. and Lactobacillus spp.) exposure during the perinatal period reduced anxiety-like behaviors induced by maternal obesity, modulated the expression of genes involved in synaptic plasticity in the prefrontal cortex in the offspring, and increased brain levels of lactate and SCFAs, which are known for their ability to regulate gene expression (van der Hee and Wells, Trends Microbiol 29, 700-12, 2021). Additionally, the multi-strain probiotic decreased inflammation, as indicated by a reduction in circulating levels of pro-inflammatory cytokine interleukin-6 (IL-6) and increased SCFA production in obese dams treated with probiotics (Radford-Smith et al., Proc Natl Acad Sci USA 119, 2022). The Radford-Smith study provides evidence of a critical role for probiotic species and their metabolites in the regulation of mood and behavior through changes in the expression of synaptic plasticity-related genes. In another study in mouse dams exposed to a pregnancy-specific dietary regimen, single-strain probiotic administration during second trimester of pregnancy, decreased anxiety-like behavior and modified cortical cytoarchitecture, with differential effects between male and female F₁ offspring (Surzenko et al., PLoS One 15, e0223395, 2020).

In a recent study, a differential impact of L. reuteri supplementation on gut community structure of MRD- versus MHFD-descendant F₂ offspring was observed, with an even more pronounced effect between females in the two cohorts which correlated with a dramatic increase in social behavior among MHFD-descendant F₂ females (Di Gesù et al., Cell Rep 41, 111461, 2022). These results reveal a relative instability of the microbiome in MHFD-descendants, especially among females, and provide rationale for targeting the maternal gut microbiome to prophylactically prevent the adverse effects of HFD during pregnancy on fetal neurodevelopment and behavioral outcomes.

While the beneficial effects of L. reuteri supplementation on offspring behavior appear to be circuit-specific and limited to the treatment of social dysfunction (Hsiao et al., Cell 155, 1451-63, 2013), it is hypothesized that a multi-strain probiotic treatment would have had a broader spectrum of efficacy and potentially synergistic effects in counteracting the detrimental effects of HFD on the maternal microbiome (Kwoji et al., Biology (Basel) 10, 2021).

Based on a thorough review of existing literature, seven species were selected with known efficacy in ameliorating metabolic, immune, and brain dysfunction. Importantly, while the vast majority of studies testing the efficacy of probiotics are focused on affected individuals or animal models of disease, the studies aimed to delineate the effects of such treatments on the ‘normal’ microbiota, in order to assess potential detrimental consequences in healthy subjects. Therefore, both regular and HFD-fed dams were treated with the probiotic cocktail starting at mating and throughout pregnancy and lactation.

Assessment of social behavior in the offspring regular or HF diet-fed females either treated or untreated with the probiotic cocktail revealed a remarkable ability of maternal probiotics in preventing the onset or mitigating the severity of social dysfunction in both males and females of the MHFD F₁ generation, without altering the social preference in MRD-descendants. These findings demonstrate the prophylactic benefits of probiotic delivery during pregnancy and support the therapeutic modulation of maternal gut microbiome in the context of diet-induced dysbiosis as a new, safe approach to improving mental health in children exposed to maternal obesity.

### I. Probiotic Compositions

Certain embodiments are directed to compositions having or comprising multiple probiotic bacteria and methods of using the same. In certain aspects the probiotic bacteria are selected form a Bifidobacterium, Lactobacillus, and Streptococcus. In particular aspects the probiotic bacteria strains are Bifidobacterium longum ATCC 55813, Bifidobacterium longum ssp. Infantis ATCC 15697, Lactobacillus acidophilus ATCC 4356, Lactobacillus paracasei ATCC 25302, Lactobacillus plantarum ATCC 14917, Lactobacillus reuteri ATCC 6475, Streptococcus salivarius subsp. thermophilus ST28, or mixtures thereof. The term “microbe” refers to an organism that is too small to be seen by the naked human eye. As used herein, the term “microbe” refers to a bacterium, a fungus, an archaea, or a protist. The term “bacteria” includes any prokaryotic organism that does not have a distinct nucleus.

Bifidobacterium longum is a Gram-positive, catalase-negative, rod-shaped bacterium present in the human gastrointestinal tract. Bifidobacterium longum as used herein can include but is not limited to one or more of the following strains: ssp. Infantis, ATCC 55813, ATCC 15697, 55814, ATCC 55815, ATCC 55816, ATCC 55817, ATCC 55818, ATCC 15707, BCRC 11847, CCRC 11847, CCUG 28903, CGMCC 1.2186, CIP 64.62, DSM 20219, E194b, HAMBI 554, JCM 1217, KCTC 3128, LMG 10497, LMG 13197, NCAIM B.01819, NCDO 2259, NCFB 2259, NCIMB 702259, or NCTC 11818.

Lactobacillus acidophilus is a species of Gram-positive bacteria that ferments sugars into lactic acid. Lactobacillus acidophilus as used herein can include one or more of the following strains ATTC 4356, NCFM, CB_LA1, La-14, FS14, or YT1.

Lactobacillus paracasei is a gram-positive, homofermentative species of lactic acid bacteria that are commonly used in dairy product fermentation. Lactobacillus paracasei as used herein can include one or more of the following strains ATCC 25302, BL23, Shirota, subsp. paracasei L1, BCRC 12188, BCRC 12248T, BCRC 14001, BCRC 14023, BCRC 16100, BCRC 17002, BCRC 17483, BCRC 17484, BCRC 17485, BCRC 17488, BCRC 17489

Lactobacillus plantarum is a bacteria commonly found in many fermented food products as well as anaerobic plant matter. Lactobacillus plantarum as used herein can include one or more of the following strains ATCC 14917, ATG-K2, ATG-K6, ATG-K8, DSM 21380, DSM 16997, DSM 6595, DSM 9843, DSM 15312, DSM 15313, or DSM 15316.

Lactobacillus reuteri is a lactic acid bacterium found in a variety of natural environments, including the gastrointestinal tract of humans and other animals. Lactobacillus reuteri as used herein can include one or more of the following strains ATCC 6475, DSM 23139, DSM 25140, DSM 25141, DSM 25683, DSM 25684, or DSM 25685.

Streptococcus salivarius is a spherical, gram-positive, facultative anaerobic bacteria that is both catalase and oxidase negative. Streptococcus salivarius as used herein can include one or more of the following strains subsp. thermophilus ST28, NS 18, NS 19, K-12, or M-18.

Another aspect of the invention relates to a composition comprising a microbial species as described above and an acceptable excipient, carrier or diluent. Suitable excipients, diluents, carriers are described below.

The composition may be any composition, but is preferably a composition to be administered orally, enterally or rectally. For example, the composition may be an edible composition. “Edible” means a material that is approved for human or animal consumption.

Another aspect of the invention relates to a probiotic composition comprising a microbial species as described above.

As used herein, the term “probiotic” means microbial cell preparations or components of microbial cells with a beneficial effect on the health or well-being of a host, including dependent organisms such as a fetus. (Salminen et al., Trends Food Sci. Technol. 1999:10 107-10).

Preferably, the probiotic composition is an orally administrable composition of metabolically active, i.e., live and/or or lyophilized, or non-viable heat-killed, irradiated or lysed probiotic microbes. The probiotic composition may contain other ingredients. The probiotic composition of the invention can be administered orally, i.e., in the form of a tablet, capsule or powder. Other ingredients (such as vitamin C, for example), may be included as oxygen scavengers. Prebiotic substrates such as these improve the colonization and survival in vivo. Alternatively, the probiotic composition of the invention may be administered orally as a food or nutritional product, such as milk or whey based fermented dairy product, or as a pharmaceutical product.

A suitable daily, weekly, or monthly dose of the probiotic microbe is from about 1×10³ to about 1×10¹¹ colony forming units (CFU), more preferably from about 1×10⁷ to about 1×10¹⁰ CFU, even more preferably, about 1×10⁶ to about 1×10¹⁰ CFU. Administered 1, 2, 3, 4, 5, 6, 7, 8, 9, 10 or times a day, week, or month.

In one preferred embodiment, the composition contains the microbial species and/or cellular components thereof, as active ingredients, in an amount of from about 1×10⁶ to about 1×10¹¹ CFU/g, respect to the weight of the composition, preferably from about 1×10⁸ to about 1×10¹⁰ CFU/g. The dose may be 0.25 g, 0.5 g, 1 g, 3 g, 5 g, 10 g or more dry weight or wet weight.

Typically, a probiotic is optionally combined with at least one suitable prebiotic compound. A prebiotic is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides.

Preferably, the composition of the present invention includes a prebiotic in an amount of from about 1 to about 30% by weight or dry weight with respect to the total weight or dry weight of the composition, preferably from 5 to 20% by weight or dry weight. Preferred carbohydrates are selected from: fructo-oligosaccharides (or FOS), short-chain fructo-oligosaccharides, inulin, isomalt-oligosaccharides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. Particularly preferred prebiotics are the short-chain fructo-oligosaccharides (for simplicity shown hereinbelow as FOSs-c.c); said FOSs-c.c. are not digestible carbohydrates, generally obtained by the conversion of the beet sugar and including a saccharose molecule to which three glucose molecules are bonded.

Feedstuffs/Products. A further aspect of the invention relates to food products, dietary supplements, nutraceuticals, nutritional formulae, drinks, and medicaments containing microbial species as defined above, and use thereof.

In one preferred embodiment, the composition comprises additionally at least one other kind of other food grade microbe, wherein the food grade microbe is preferably selected from the group consisting of one or more of seven probiotic bacterial strains (Bifidobacterium longum ATCC55813, Bifidobacterium longum ssp. Infantis ATCC 15697, Lactobacillus acidophilus ATCC 4356, Lactobacillus paracasei ATCC 25302, Lactobacillus plantarum ATCC 14917, Lactobacillus reuteri ATCC 6475, Streptococcus salivarius subsp. thermophilus ST28), or mixtures thereof. In certain aspects the probiotic can be in a dry or semi dry state and added to a delivery vehicle such as a liquid or food stuff prior to being eaten, drank, or otherwise administered.

One aspect of the invention relates to a food product comprising the microbial species defined above. The term “food product” is intended to cover all consumable products that can be solid, jellied or liquid. Suitable food products may include, for example, functional food products, food compositions, pet food, livestock feed, health foods, feedstuffs and the like. In one preferred embodiment, the food product is a health food or a nutritional supplement.

As used herein, the term “functional food product” means food that provides not only a nutritional effect but is also capable of delivering a further beneficial effect to the consumer. Accordingly, functional foods are ordinary foods that have components or ingredients (such as those described herein) incorporated into them that impart to the food a specific functional—e.g., medical or physiological benefit—other than a purely nutritional effect.

Examples of specific food products that are applicable to the present invention include milk-based products, ready to eat desserts, powders for re-constitution with, e.g., milk or water, chocolate milk drinks, malt drinks, ready-to-cat dishes, instant dishes or drinks for humans or food compositions representing a complete or a partial diet intended for pets or livestock.

In one preferred embodiment the composition according to the present invention is a food product intended for humans, pets, or livestock. The composition may be intended for animals selected from the group consisting of dogs, cats, pigs, cattle, horses, goats, sheep, or poultry. In a preferred embodiment, the composition is a food product intended for adult species, in particular human female adults, and more particularly those female adults attempting to get pregnant or are already pregnant.

In the present invention, “milk-based product” means any liquid or semi-solid milk or whey-based product having a varying fat content. The milk-based product can be, e.g., cow's milk, goat's milk, sheep's milk, skimmed milk, whole milk, milk recombined from powdered milk and whey without any processing, or a processed product, such as yogurt, curdled milk, curd, sour milk, sour whole milk, butter milk and other sour milk products. Another important group includes milk beverages, such as whey beverages, fermented milks, condensed milks, infant or baby milks; flavored milks, ice cream; milk-containing food such as sweets.

One aspect of the invention relates to a feedstuff or animal feed comprising the microbial species defined above.

The compositions of the present invention may be—or may be added to—food supplements, also referred to herein as dietary or nutritional supplements or food additives. Thus, another aspect of the invention relates to a dietary supplement or food additive comprising one or more microbial strains according to the invention.

Diluents, Excipients and Carriers. As mentioned above, the invention also relates to compositions, more preferably pharmaceutical compositions, or nutritional supplements, comprising the microbial species compositions defined above, and use thereof. The microbial species is generally administered in admixture with a pharmaceutically or nutritionally acceptable carrier, excipient, or diluent, particularly for human therapy. The pharmaceutical compositions may be for human or animal usage in human and veterinary medicine.

Examples of such suitable excipients for the various forms of pharmaceutical compositions described herein may be found in the “Handbook of Pharmaceutical Excipients, 2nd Edition, (1994), Edited by A Wade and P J Weller. Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilizing agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Nutritionally acceptable carriers, diluents and excipients include those suitable for human or animal consumption and that are used as standard in the food industry. Typical nutritionally acceptable carriers, diluents and excipients will be familiar to the skilled person in the art.

Administration. The compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration. Preferably, the compositions of the present invention are adapted for oral, buccal, or sublingual routes of administration. For oral administration, particular use is made of compressed tablets, pills, tablets, gellules, drops, powders, foodstuffs, and capsules. Compositions may be formulated in unit dosage form, i.e., in the form of discrete portions containing a unit dose, or a multiple or sub-unit of a unit dose.

Dosage. A person of ordinary skill in the art can easily determine an appropriate dose of one of the instant compositions to administer to a subject without undue experimentation. Typically, a physician or other person preparing a composition for administration will determine the actual dosage which will be most suitable for an individual and it will depend on a variety of factors including the activity of the specific microbial strain(s) employed, the metabolic stability and length of action of that strain or strains, the age, body weight, general health, sex, diet, mode and time of administration, rate of excretion, drug combination, the severity of the particular condition, and the individual undergoing therapy. The dosages disclosed herein are an example of the average case. There can of course be individual instances where higher or lower dosage ranges are merited, and such are within the scope of this invention.

The usual effective daily dose in humans is from about 1×10³ to about 1×10¹¹, more preferably, from about 1×10⁷ to about 1×10¹¹, even more preferably, from about 1×10⁶ to about 1×10¹⁰ CFU.

Combinations. In a particularly preferred embodiment, the compositions of the invention are administered in combination with one or more other active agents. In such cases, the compositions of the invention may be administered consecutively, simultaneously or sequentially with one or more other active agents. The probiotic compositions described herein can be administered in combination with maternal supplements. “Maternal supplement” refers to a composition formulated for administration to a pregnant or lactating female. A maternal supplement may be provided in the form of a pill or tablet or may be provided as a functional food or beverage. A maternal can include a protein, a vitamin, a mineral, or a combination of any of the foregoing. Non-limiting examples of proteins include caseins, alpha-lactalbumin, and/or lactoferrin. Non-limiting examples of vitamins and minerals include iron, vitamin B6, vitamin B9, vitamin B12, calcium, magnesium, phosphorus, iron, zinc, copper, iodine, selenium, vitamin A or retinol activity equivalent (RAE) e.g. beta carotene or a mix of carotenoids, Vitamin C, Vitamin B1, niacin, folic acid, biotin, tocopherol, selenium.

In addition to proteins and/or vitamin and/or minerals, maternal supplement may also include: lipids, carbohydrates, pharmaceutically active agents and, conventional additives such as anti-oxidants, stabilizers, emulsifiers, acidulants, thickeners, buffers or agents for pH adjustment, chelating agents, colorants, excipients, flavor agents, osmotic agents, pharmaceutically acceptable carriers, preservatives, sugars, sweeteners, texturizers, emulsifiers, and water. It may be particularly beneficial if the maternal supplement includes lipids, e.g., Long chain polyunsaturated fatty acids. Some of these compounds are believed to impact the risk of preterm birth.

### II. Examples

The following examples as well as the figures are included to demonstrate certain embodiments of the invention. It should be appreciated by those of skill in the art that the techniques disclosed in the examples or figures represent techniques discovered by the inventors to function well in the practice of the invention, and thus can be considered to constitute a mode for its practice. However, those of skill in the art should, in light of the present disclosure, appreciate that many changes can be made in the specific embodiments which are disclosed and still obtain a like or similar result without departing from the spirit and scope of the invention.

### Example 1

### Probiotic-Mediated Modulation of the Maternal Gut Microbiome During Pregnancy Prevents Social Dysfunction in the Offspring of HFD-Fed Dams

### A. Materials and Methods

Mice and maternal diet. WT C57BL/6N mice were obtained from Taconic Laboratories (B6), maintained at animal biosafety level-1 under specific pathogen free (SPF) conditions, and kept on a 12-hour light/dark cycle in polysulfone cages with access to sterile food and water ad libitum. Six-week-old females were placed on either a regular diet (RD) consisting of 13.4% kcal from fat, 30% kcal from protein, and 57% kcal from carbohydrates (Lab Diets, #5001) or high-fat diet (HFD) consisting of 60% kcal from fat, 20% kcal from protein, and 20% kcal from carbohydrates (Research Diets, #D12492). After six-to-eight weeks diet, females were randomly assigned to monogamous pairs with WT C57BL/6N adult males to produce subject offspring. Probiotic cocktail (PC) was administered in drinking water daily through pregnancy and lactation. Four P generation maternal groups, HFD, HFD+PC, RD, RD+PC, were obtained, and male and female F₁ descendants—weaned at 3 weeks of age onto RD chow, regardless of maternal diet (RD or HFD) —were classified as MHFD, MHFD MPC, MRD, and MRD MPC. Behavioral tests were performed at 7 to 8 weeks of age, with handling for three days prior in conjunction with stool collection. Animal care and experimental procedures were approved by The University of Texas Medical Branch Institutional Animal Care and Use Committee in accordance with all guidelines set forth by the U.S. National Institutes of Health.

Behavior. Behavioral assays were performed as previously described (Buffington et al. Cell 165, 1762-75, 2016), as detailed in brief below. Animals were acclimated to the behavioral suite for 30 to 60 minutes prior to initiation of all experiments. Apparatuses were spot cleaned with 70% ethanol after each animal and thoroughly cleaned at the end of the day. ANY-maze automated video tracking system software version 6.33 (Stoelting) was used for video recording and automated data acquisition.

Reciprocal social interaction. Mice were placed in a neutral, 30 cm³ Plexiglass arena containing a shallow layer of corncob bedding with a previously unencountered age- and sex-matched conspecific matched for maternal diet. Time spent engaged in social interaction (close following, touching, nose-to-nose sniffing, nose-to-anus sniffing, and/or crawling over/under each other) over a ten-minute period was recorded by a blinded human observer and analyzed via ANY-maze.

Crawley's three-chamber (3C) test for sociability and preference for social novelty. Crawley's 3C test for sociability and preference for social novelty was performed as described (Silverman et al., Nat Rev Neurosci 11, 490-502, 2010). Briefly, animals were habituated for 10 minutes in a 60×40×23 cm arena divided into three inter-connected chambers. Sociability was measured during a second 10-minute interval in which the test subject could interact either with an empty wire cup (Empty) or a wire cup containing an age- and sex-matched stranger conspecific (Mouse 1). Stranger mice were acclimated to restraint in the wire cup during three training periods, over a period of two days prior to the test. Preference for social novelty was assayed by introducing a second stranger mouse (Mouse 2) into the previously empty wire cup. The location of the Empty Cup/Mouse 2 versus Mouse 1 were counterbalanced within cohorts. Interactions were again recorded by ANY-maze software and an independent observer.

Open field test. Animal test subjects were gently lowered into an open arena (40×40×20 cm) and allowed to explore freely for 10 minutes. ANY-maze automatically measured distance traveled, speed, and position in the arena, as well as time spent in the center of the arena (defined as the interior 20×20 cm).

16S ribosomal RNA (rRNA) gene amplicon (metataxonomic) sequencing. Stool samples were aseptically collected in sterile 2 mL Eppendorf tubes, immediately placed on dry ice, and stored at −80° C. until further processing. Bacterial DNA was extracted and sequenced by the Alkek Center for Metagenomics and Microbiome Research at Baylor College of Medicine using protocols adapted from those developed for the NIH-Human Microbiome Project (Human Microbiome Project, Nature 486, 215-221, 2012), as described previously (Buffington et al. Cell 165, 1762-75, 2016). Briefly, bacterial genomic DNA was extracted using MagAttract PowerSoil DNA Kit (Qiagen) followed by PCR amplification of the 16S rDNA V4 region. The primers used for amplification include MiSeq adapters and single-end barcodes allowing for pooling and direct sequencing of PCR products. Sequencing was performed on the Illumina MiSeq platform using the 2×250 bp paired-end protocol yielding overlapping paired-end reads. The 16S rRNA gene read pairs were demultiplexed and merged using USEARCH v7.0.1090 (Edgar, Bioinformatics 26, 2460-61, 2010), allowing zero mismatches and a minimum overlap of 50 bases. Merged reads were trimmed at first base with Q5. A quality filter was applied to the resulting merged reads and reads containing above 0.05 expected errors were discarded. 16S rRNA gene sequences were clustered into Operational Taxonomic Units (OTUs) at a similarity cutoff value of 97% using the UPARSE algorithm (Edgar, Nat Methods 10, 996-98, 2013). OTUs were mapped to an optimized version of the SILVA Database (Quast et al., Nucleic Acids Res 41, D590-96, 2013) containing only the 16S v4 region to determine taxonomies. Abundances were recovered by mapping the demultiplexed reads to the UPARSE OTUs. A custom script constructed a rarefied OTU table from the output files generated in the previous two steps for downstream analyses of alpha-diversity, beta-diversity, and phylogenetic trends using the Agile Toolkit for Incisive Microbial Analyses (ATIMA2) platform (URL atima.research.bcm.edu).

Limosilactobacillus (L.) reuteri culture and treatment. L. reuteri PTA-6475 was purchased from ATCC (MM4-1A). Cultures were grown anaerobically in De Man, Rogosa, and Sharpe (MRS) broth (MilliporeSigma) for 48 hours in a 5% CO₂, 5% H₂, remainder N₂ environment. Stocks were aliquoted and frozen at −80° C. then moved to −20° C. storage the week of use. Stock viability was confirmed and colony forming units (CFU) quantified by serial plating on MRS and growing anaerobically for 48 hours. For treatment of F₂ offspring, 200 mL L. reuteri was added to 200 mL sterile drinking water at a concentration of 1×10⁸ CFU/mL at time of weaning. Treated water was refreshed daily, Monday-Friday, for at least four weeks prior to behavior, and was continued throughout behavioral assessment until time of tissue collection.

Multi-strain probiotic culture and treatment. Cultures of seven probiotic bacterial strains (Bifidobacterium longum ATCC55813, Bifidobacterium longum ssp. Infantis ATCC 15697, Lactobacillus acidophilus ATCC 4356, Lactobacillus paracasei ATCC 25302, Lactobacillus plantarum ATCC 14917, Lactobacillus reuteri ATCC 6475, Streptococcus salivarius subsp. thermophilus ST28), selected by literature review for beneficial, immunomodulatory taxa, were cultured anaerobically in de Man, Rogosa & Sharpe (MRS) broth (Millipore Sigma #110661) or Brain heart infusion (BHI) broth (Millipore Sigma #110493), for 24 hours, at 37° C., in a 5% CO₂, 5% H₂, 90% N₂ gas mixture atmosphere. Cultures were then centrifuged at 3,000 rpm for 10 minutes at 4° C. to pellet, after which time, media supernatant was removed. The resulting pellets were resuspended in sterile, anaerobic PBS to a final concentration of OD600 nm=50 and aliquoted into 15 ml conical tubes. Bacterial stocks were stored individually at −80° C. and moved to −20° C. storage the week of use.

For treatment of either RD- or HFD-fed dams, equal volumes of each of seven strains were thawed and combined into one cocktail concentrate at 10⁹ CFU/mL. To 200 mL of sterile drinking water was added 400 μL of probiotic cocktail, to yield a final concentration of OD600 nm=0.1. Probiotic cocktail-laced water was replaced each day, Monday through Friday, starting at the time of breeding and continuing throughout pregnancy and lactation.

Statistics. Power analyses to establish group size were performed in GPower 3.1. Data were analyzed and visualized using GraphPad Prism version 8.4.3 for Mac OS X, GraphPad Software, San Diego, California USA. Data are presented as mean±SEM unless otherwise indicated. Statistics are reported in figure legends as t(degrees of freedom)=t-statistic/q-statistic, p=p-value. p<0.05 was considered significant, where *p<0.05, **p<0.01, ***p<0.001, and ****p<0.0001. For data without repeated measures (3C locomotor data and open field test data), data were visualized as column data but analyzed using a two-tailed nested t-test to account for cage effects. No significant differences between sex-matched cages within the same cohort were identified. For Crawley's 3C assay, a mixed-effects model with repeated measures was applied, with the Sidak correction for multiple comparisons (Lazic and Essioux, BMC Neurosci 14, 37, 2013; Jimenez and Zylka, J Neurodev Disord 13, 2, 2021). Random (litter) effects with a variance of zero were removed from the model and analyzed by repeated measures two-way ANOVA. For reciprocal social experiments, an unpaired, two-tailed t-test was performed unless otherwise indicated. In cases where data were not normally distributed, the non-parametric Mann-Whitney test was used. For germ-free mice, two litters per group were used. Locomotor data was analyzed by one-way ANOVA with Tukey's adjustment for multiple comparisons. 3C results were analyzed as above. Social Preference Indexes were calculated as in (Rein et al., Nat Protoc 15, 3464-77, 2020) and analyzed using a nested t-test. Differentially abundant bacterial taxa were determined by Linear Discriminant Analysis (LDA) Effect Size (LEfSe) (Segata et al., Genome Biol 12, R60, 2011) using the Galaxy computational tool (Afgan et al., Nucleic Acids Res 46, W537-W544, 2018) and applying the all-against-all strategy with a minimum logarithmic LDA score (i.e., biomarker effect size) of 2.0.

Summary of outliers and excluded data. A limited number of animals were removed from analysis due to statistical identification as outliers and/or failure to meet pre-established criteria. Outliers were identified by Grubbs' test (alpha=0.05, URL graphpad.com/quickcalcs/grubbs1/) and were removed from the dataset (e.g., OF, RS, or 3C). For 3C, Grubbs' test was run on the difference between interaction times (M₁−E, and M₂−M₁). Additionally, mice that failed to interact for a minimum of 10 seconds with either social or non-social object in the 3C task were removed from the entire 3C dataset.

### B. Results

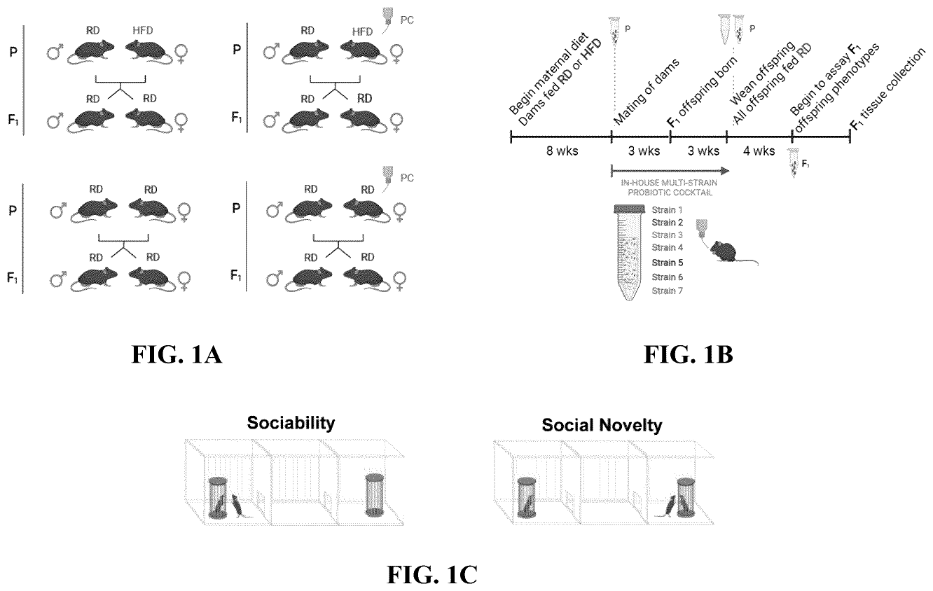

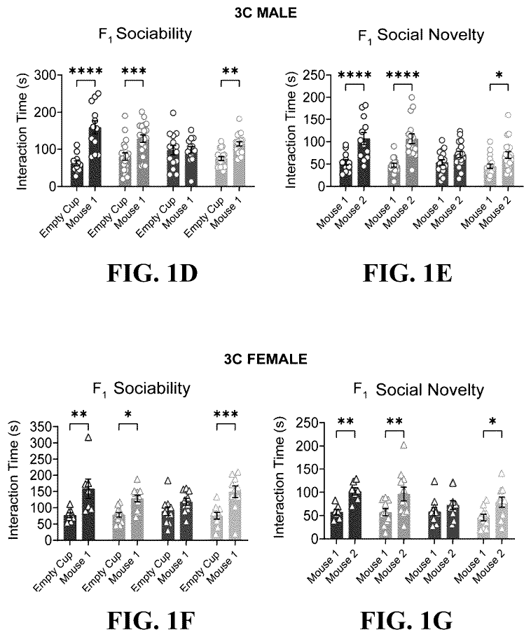

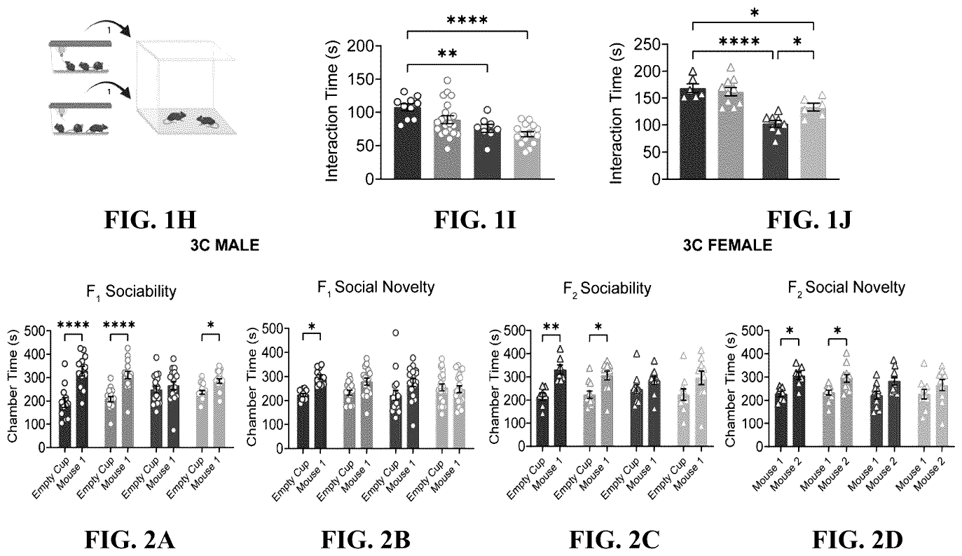

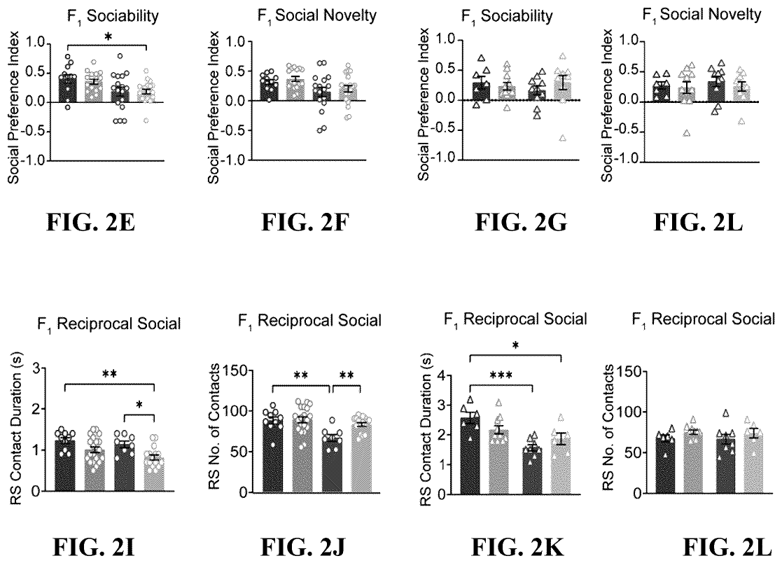

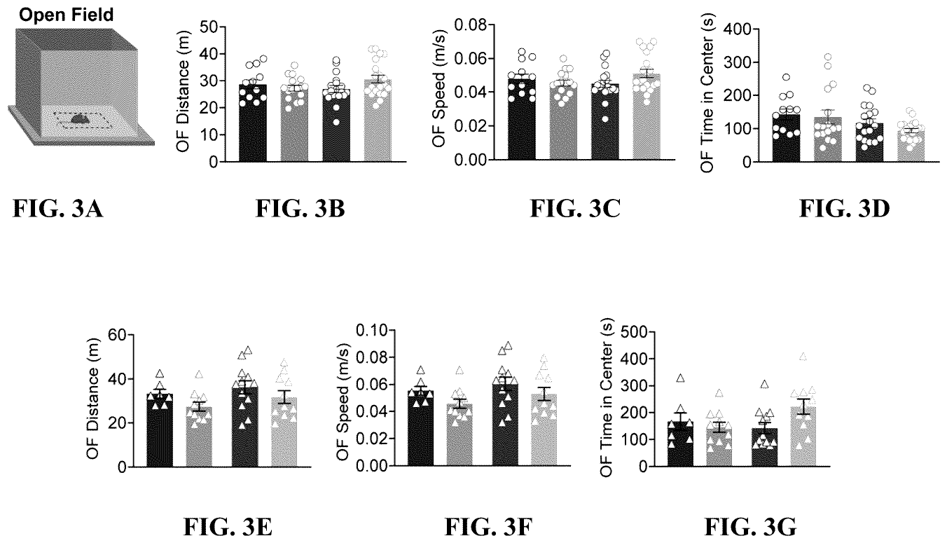

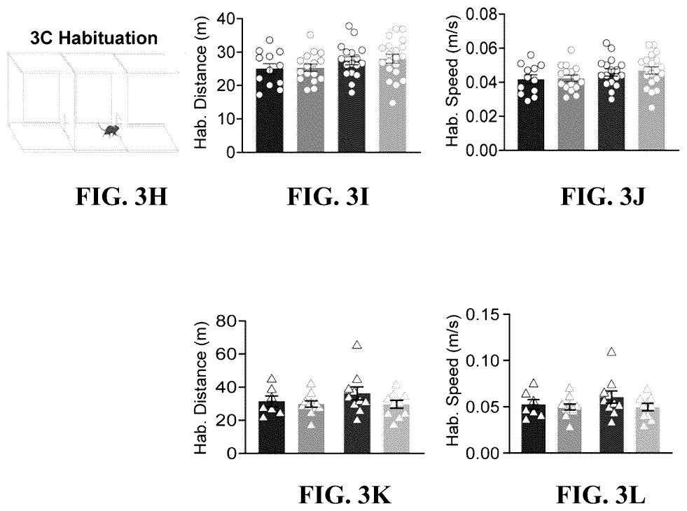

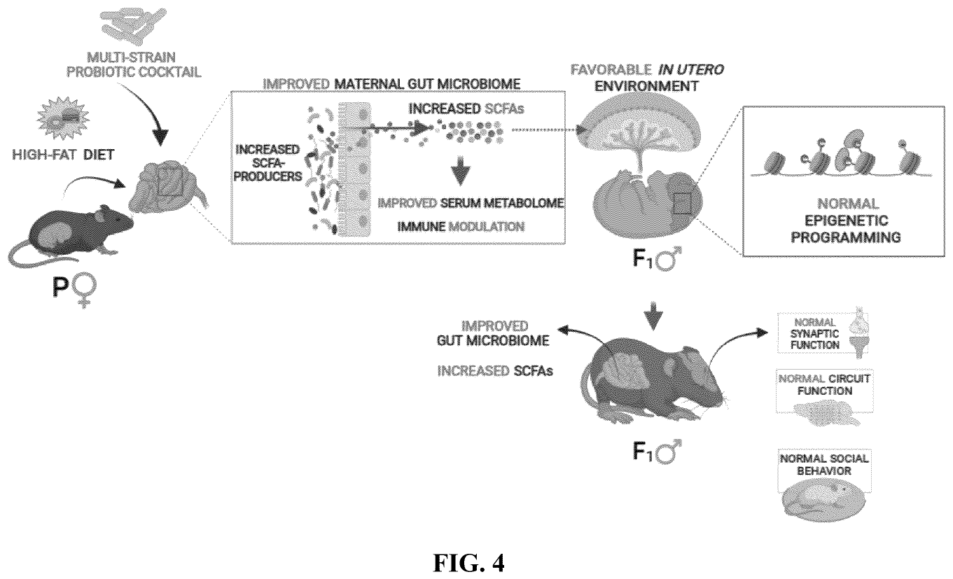

Maternal probiotic treatment during pregnancy and lactation prevents social dysfunction in the offspring of HFD-fed dams. Detrimental effects of pre- and early post-natal exposure to MHFD on offspring brain function and behavior have been reported by multiple investigations, including ours (Buffington et al., Cell 165, 1762-75, 2016; Bilbo and Tsang, FASEB J 24, 2104-15, 2010; Fernandes et al., Transl Psychiatry 11, 149, 2021). Exposure to prenatal probiotics aimed at correcting maternal diet-induced dysbiosis of the maternal and offspring microbiota has been proposed as a new therapeutic opportunity to prevent the onset of behavioral disorders in the offspring (Radford-Smith et al., Proc Natl Acad Sci USA 119, 2022). The inventors sought to determine whether social deficits observed in the F₁ generation could be prevented by maternal administration of a multi-strain probiotic cocktail starting at mating and throughout pregnancy and lactation. To test the hypothesis, C57Bl/6N females were fed a regular diet (RD) or HFD for eight weeks prior to mating to produce the F₁ generation male and female offspring (FIG. 1A). Assigned preconception maternal diet was maintained throughout pregnancy and lactation, and probiotic cocktail was administered in drinking water daily through pregnancy and lactation (FIG. 1B). Four P generation maternal groups, HFD, HFD+PC, RD, RD+PC, were obtained, and male and female F₁ descendants—weaned onto RD chow, regardless of maternal diet—were classified as MHFD, MHFD MPC, MRD, and MRD MPC.

Stool samples were collected from the P generation dams after 8 weeks on diet (i.e., prior to mating), and after pup weaning, to characterize the effect of the probiotic cocktail on gut microbial ecology in future experiments using 16S ribosomal RNA (rRNA) gene amplicon (metataxonomic) sequencing. Together with serum samples, a second set of fecal samples was collected to analyze diet and probiotic effects on the maternal metabolome and cytokine expression (FIG. 1B).

The F₁ mice were caged according to sex, maternal lineage, and maternal treatment at weaning, then assessed social and anxiety-like behavior in 7-to-10-week-old male and female HFD, HFD+PC, RD, RD+PC F₁ descendants. Stool samples were collected from the F₁ cohorts at the time of behavior to characterize the gut microbiome community structure of the RD versus HFD F₁ subjects, with or without probiotic supplementation (FIG. 1B).

MHFD, MHFD MPC, MRD, and MRD MPC F₁ cohort were assessed for social behavior using Crawley's 3C test for sociability and preference for social novelty (FIG. 1C). Male and female behavior were analyzed separately to allow for identification of sexually dimorphic phenotypes upon maternal probiotic treatment, given the differential effect of post-weaning L. reuteri treatment in F₂ offspring. Consistent with previous findings, while MRD-descendant F₁ males showed strong sociability and preference for social novelty, MHFD-descendant F₁ males showed significant impairments. Remarkably, the deficits in social behavior were prevented by maternal probiotic treatment (FIGS. 1D, 1E). Unexpectedly, deficits in both sociability and preference social novelty were observed in MHFD-descendant F₁ females compared to their MRD counterparts, which were also corrected by maternal probiotic cocktail administration (FIGS. 1F, 1G).

The assessment of reciprocal social interaction between sex-matched stranger pairs (FIG. 1H) revealed that MHFD reduced the time F₁ female pairs spent interacting compared to MRD-descendant F₁ female pairs. Maternal probiotic cocktail was effective in increasing MHFD-descendant interaction time, which remained low relative to MRD cohorts (FIG. 1J). While no differences were observed in the number of contacts during the reciprocal social interaction task across all experimental groups, MHFD reduced contact duration in the F₁ females, with no major effect of maternal probiotic treatment.

Consistent with previous results, MHFD-descendant F₁ male pairs spent significantly less time interacting than MRD-descendant F₁ male pairs, regardless of maternal probiotic cocktail administration (FIG. 1I). While the number of contacts were decreased by MHFD and increased by maternal probiotic cocktail administration, contact duration was significantly decreased in MHFD MPC F₁ male pairs. Collectively, these data suggest a strong social dysfunction phenotype in both male and female MHFD-descendants, with maternal probiotic treatment exerting a protective effect against the social deficits induced by early-life exposure to MHFD.

In line with previous findings in the F₂ generation offspring, based on the results of the open field test to assess hyperactivity and anxiety-like behavior in the F₁ generation, there was no main effect of maternal diet (HF or control) and no maternal diet×treatment interaction on locomotor activity, as measured by distance traveled and speed, regardless of sex. These results matched those obtained by the habituation phase of the 3C task for both males and females. Similarly, neither maternal diet or probiotic treatment impacted anxiety-like behavior in the open field test in the juvenile male and female F₁ offspring, as measured by time spent in the center of the open field arena. Taken together, these results indicate that social impairments observed in MHFD-descendant F₁ males and females, which are rescued by maternal probiotic treatment, (FIG. 1) are not related to anxiety-like or hyperactivity phenotypes.

The maternal microbiome plays a crucial role in both fetal and early postnatal neurodevelopment. Environmental factor-driven dysbiosis in maternal gut communities are associated with deficits in social function in both humans and animal models. Pregnancy determines significant and progressive changes in maternal physiology at the metabolic, immune, and gastrointestinal levels, including robust weight gain, insulin resistance, and increased inflammatory response. These changes are especially pronounced in the third trimester to accommodate for the increased energy requirements of the growing fetus. The modifications are accompanied by substantial rearrangements in the composition and the function of the gut microbiome, which have been suggested to occur as the result of the cross-talk between the gut microbial communities and the endocrine system (Mallott et al., Sci Rep 10, 9961, 2020; Flores et al., J Transl Med 10, 253, 2012). Recent experimental evidence identifies the microbiome as a primary mediator of the remodeling of the maternal environment during pregnancy aimed to ensure optimal fetal growth and development (Di Simone et al., Front Immunol 11, 528202, 2020), however, the underlying molecular and cellular mechanisms are understudied and remain unclear (Yeo et al., J Endocrinol 253, R1-R19, 2022).

Given the role of environmental factors, such as antibiotic administration, lifestyle, and dietary habits, in modifying the gut microbiome, investigation is underway into how these factors, especially maternal diet, may interfere with the physiological evolution of the maternal microbiome as pregnancy progresses. Maternal obesity and overnutrition may exacerbate or impede the progression of gut microbial adaptation in normal pregnancy, and thereby impact maternal metabolic and immunological changes, ultimately creating sub-optimal environment intrauterine environment and predisposing the fetus to obesity, metabolic dysfunction, and/or diabetes later in life (Dreisbach et al., Front Cell Infect Microbiol 11, 795924, 2021; Paul et al., Mol Nutr Food Res 62, 2018).

Maternal interventions that address diet-induced dysbiosis of the gut microbiome, such as supplementation with pre- or probiotics, might represent a promising new avenue to prevent maternal programming of offspring disease susceptibility. For instance, supplementation with prebiotics during pregnancy ameliorated metabolic dysfunction in rats exposed to high-fat/high-sucrose diet and reduced offspring risk for obesity (Paul et al., Sci Rep 6, 20683, 2016). Likewise, maternal multi-strain probiotic treatment ameliorated lipid and glucose profiles, and modified levels of metabolic hormones in the offspring of HFD-fed females while exerting a positive effect on offspring gut dysbiosis (Guo et al., Food Funct 9, 4317-27, 2018).

As discussed in the introduction, there is a scarcity of studies focusing on the effect of maternal pre- and probiotics on disorders of brain function and behavior in the offspring resulting from exposure to MHFD. The inventors show that maternal HFD regimen starting eight weeks prior to mating and maintained through pregnancy and lactation drove social deficits in F₁ male offspring (FIGS. 1D, 1E, 1J). Additionally, similar impairments were observed in in MHFD F₁ females, as demonstrated by reduced sociability (FIG. 1F), loss of preference for social novelty (FIG. 1G), and reduced interaction time in the reciprocal social interaction task (FIG. 1J), in partial agreement with previous studies (Bordeleau et al., Brain Behav Immun Health 15, 100281, 2021).

In F₁ male cohorts, it was found that maternal supplementation with a multi-strain probiotic during pregnancy and lactation was sufficient to normalize MHFD-descendant F₁ sociability, with a weaker, yet statistically significant, effect on preference for social novelty and reciprocal social interaction, thus revealing a differential effect of prenatal probiotics on various aspects of social behavior, which are regulated by distinct neuronal circuits (Chen et al., Nature 586, 270-74, 2020). Conversely, among F₁ female groups, maternal probiotic treatment showed comparable efficacy in preventing the detrimental effect of the maternal HFD regimen on descendant sociability, preference for social novelty, and reciprocal social interaction between stranger pairs.

The data contradict a recent report showing no effect of maternal probiotics on preventing social dysfunction in the offspring of HFD-fed dams on either 8-week-old male or female subjects (Radford-Smith et al., Proc Natl Acad Sci USA 119, 2022). Notably, only a single test was used to assess social behavior in the Radford-Smith et al. study and they used a different probiotic cocktail. This discrepancy is important to note given that specific probiotics affect particular neuronal circuits—e.g., while Bacteroides fragilis corrects communication deficits and stereotypic behavior in a common mouse model for maternal immune activation-induced ASD, it was shown to have no impact on social deficits (Hsiao et al., Cell 155, 1451-63, 2013). Additionally, the assessment of anxiety-like behavior in the offspring showed no effect of maternal diet or probiotic treatment, while in (Radford-Smith et al., Proc Natl Acad Sci USA 119, 2022) MHFD-induced decrease in time spent in the center of the open field was prevented by maternal probiotic treatment. However, these differences in anxiety-like behavior might be due to the different subject age at the time of the test, eight weeks in our study versus three weeks. Another factor to consider is the different mouse strain used, inbred C57BL/6N vs outbred CD-1 IGS, which might influence both MHFD-induced behavioral deficits in offspring and the effects to maternal probiotic treatment. Indeed, recent studies reported strain-specific effects of probiotic administration (Casaro et al., Microbiome 9, 134, 2021), potentially due to distinct microbiota configurations based on mouse genetic background (Campbell et al., ISME J 6, 2033-44, 2012).

Lastly, the effects of probiotics on microbial communities of the gut and different clinical and mechanistic outcomes are both strain- and dose-dependent (Stenman et al., Behav Brain Res 379, 112376, 2020; Sanders et al., Ann NY Acad Sci 1309, 1-18, 2014). In the case of commercial multi-strain formulations, manufacturing factors, such as growth conditions and substrates, processing and storage conditions, might affect probiotic properties and effects (Trinchieri et al., Front Immunol 8, 1474, 2017).

In recent work, the inventors showed that MHFD F₁ females had a strong reduction in multiple SCFA-producing taxa compared to their MRD counterparts. Additionally, L. reuteri supplementation in the F₂ offspring drove expansion of SCFA producers, such as Ruminococcus and Lachnospiraceae in both MRD and MHFD descendants, identifying the L. reuteri-mediated increase in biosynthetic potential for SCFAs as potential mechanism for the associated behavioral rescue observed in F₂ MHFD offspring. This finding is supported by previous studies demonstrating the ability of Lactobacillus strains to boost intestinal levels of SCFAs (Nagpal et al., Sci Rep 8, 12649, 2018) and the neuroprotective effects of the SCFA-producing taxa in neurological conditions (Cheng et al., Crit Rev Food Sci Nutr, 1-31, 2021).

It is contemplated that a similar increase in maternal and/or offspring SCFA-producing taxa might be the primary driver of the preventative effect of the 7-strain maternal probiotic developed and use in these studies on offspring social dysfunction.

1. A multi-strain probiotic composition comprising Bifidobacterium, Lactobacillus, and Streptococcus bacteria and a prebiotic component.

2. The composition of claim 1, comprising Bifidobacterium longum, Lactobacillus acidophilus, Lactobacillus paracasei, Lactobacillus plantarum, Lactobacillus reuteri, and Streptococcus salivarius.

3. The composition of claim 2, comprising a probiotic in a ratio of 1 to 20 Bifidobacterium longum: 1 to 10 Lactobacillus acidophilus: 1 to 10 Lactobacillus paracasei: 1 to 10 Lactobacillus plantarum: 1 to 10 Lactobacillus reuteri: 1 to 10 Streptococcus salivarius.

4. The composition of claim 1, comprising Bifidobacterium longum ATCC55813, Bifidobacterium longum ssp. Infantis ATCC 15697, Lactobacillus acidophilus ATCC 4356, Lactobacillus paracasei ATCC 25302, Lactobacillus plantarum ATCC 14917, Lactobacillus reuteri ATCC 6475, and Streptococcus salivarius subsp. thermophilus ST28.

5. The composition of claim 4, wherein the Bifidobacterium longum ATCC55813, Bifidobacterium longum ssp. Infantis ATCC 15697, Lactobacillus acidophilus ATCC 4356, Lactobacillus paracasei ATCC 25302, Lactobacillus plantarum ATCC 14917, Lactobacillus reuteri ATCC 6475, and Streptococcus salivarius subsp. thermophilus ST28 are present in a ratio of 1 to 10:1 to 10:1 to 10:1 to 10:1 to 10:1 to 10:1 to 10.

6. The composition of claim 1, wherein each probiotic bacteria are present at 1×10³ to about 1×10¹¹ colony forming units (CFU) per gram.

7. The composition of claim 1, wherein the prebiotic is a non-digestible carbohydrate.

8. The composition of claim 1, wherein the composition is an edible composition.

9. The composition of claim 1, wherein the composition is in the form of a tablet, a powder, a capsule, or a solution.

10. A nutritional supplement comprising a multi-strain probiotic composition of claim 1.

11. A nutraceutical comprising a multi-strain probiotic composition of claim 1.

12. A method of treating dysbiosis of a maternal gut biome comprising administering to a female an effective amount of a multi-strain probiotic composition of claim 1.

13. A method of ameliorating the detrimental effects of a high fat diet on a fetus, infant, or fetus and infant by administering to a mother carrying or given birth to the fetus/infant an effective amount of a multi-strain probiotic composition of claim 1.
